(19) United States
(12) Patent Application Publication
(10) Pub. No.: US 2019/0365921 A1
(43) Pub. Date: Dec. 5, 2019

(54) Method and System of Harvesting Extracellular Vesicles Using Hydrogel Particles for Later Delivery to, and Remodeling of, an Immune System

(71) Applicants: GEORGE MASON UNIVERSITY, FAIRFAX, VA (US); CERES NANOSCIENCES, INC., MANASSAS, VA (US)

(72) Inventors: LANCE A. LIOTTA, BETHESDA, MD (US); ALESSANDRA LUCHINI KUNKEL, BURKE, VA (US); BENJAMIN LEPENE, LEESBURG, VA (US); FATAH KASHANCHI, ROCKVILLE, MD (US)

(21) Appl. No.: 16/430,300

(22) Filed: Jun. 3, 2019

Related U.S. Application Data
(60) Provisional application No. 62/679,524, filed on Jun. 1, 2018.

(51) Int. Cl. A61K 47/69; A61K 9/127; A01K 67/027

(52) U.S. Cl. CPC A61K 47/6903; A61K 9/1271; A01K 2267/0331; A01K 67/0278; A01K 2227/105; A61K 47/6911

(57) ABSTRACT

A system and method configured to achieve hydrogel particle treatment for the reduction of metastatic burden in an immunocompetent syngeneic mouse model is described. The system seeks to achieve methodic optimization for the extraction and purification of extracellular vesicles (EVs) from cultured cells as well as from fresh breast cancer interstitium. Similarly, the system provides for lymph node remodeling by human breast cancer EVs in a humanized mouse model. Hydrogel particles are employed to convey cytokine releasing and EV-displaying treatment to afflicted bodies. The process and system is envisioned to be applied to other diseases and cancers, and is not therefore limited to metastatic cancers

### CONTINUITY

This application is a non-provisional application of provisional patent application No. 62/679,524, filed on Jun. 1, 2018, and priority is claimed thereto.

### FIELD OF THE PRESENT INVENTION

The present invention relates to the field of medical treatment regimens via the remodeling of an immune system, and more specifically relates to the use of hydrogel particles to harvest extracellular vesicles, including exosomes, for delivery to an immune system, whereby the immune system is remodeled for therapeutic purposes.

### BACKGROUND OF THE PRESENT INVENTION

Many cancers and diseases lack adequate treatment options which can eliminate risks of mortality associated with such cancers and diseases. Many current treatment options are toxic in and of themselves, which can adversely impact survival of the patient. If there were a way in which treatment regimens for such cancers and diseases could be revolutionized via the use of hydrogel particles for the remodeling of an immune system, mortality risks could be reduced or eliminated, treatments would be both more effective and less risk prone.

As such, the immunotherapy goal of the system and method of the present invention, directed at the immune system, is to use multi-function particle technology to prime or awaken systemic immune recognition/rejection of metastatic breast cancer colonies and other types of cancers or infectious diseases.”.

While some competing treatment methods are presently available on the market, no method previously has used the remodeling function for the lymph node to achieve experimental therapeutic efficacy in an immunocompetent animal model for both cancer and infectious disease. Additionally, no other method previously has shown that exosomes loaded on any type of particle and then delivered to immune cells cause activation of the immune cells compared to the particles alone. Likewise, no other method on the market is using particles in the lymph system to sample the lymph contents, including exosomes, proteins, or other biomolecules, or to home to and deliver a plurality of factors to the lymph node that are chemo-attractants for immune cells.

Thus, there is a need for a new system and array of treatment options which employ remodeling of the immune system, including the lymph nodes, to achieve experimental therapeutic efficacy for cancer and disease. Such a system and method preferably employs hydrogel-particle-exosome harvesting with mass spectrometry to discover exosome proteins specific to cell types or disease states.

As such, hydrogel particles can now be made of multiple forms for increased surface area for binding to analytes like exosomes, similar to an artificial macrophage.

### SUMMARY OF THE PRESENT INVENTION

The system and method of the present invention purifies exosomes with hydrogel particles or with a combination of hydrogel particles and a size selection method, as described below.

Remodeling the lymph node happens by subcutaneous injection of hydrogel particles with cytokine, or loaded with exosomes. Reduction to practice data shows that a) the lymph node is completely remodeled in its cell population and b) dendritic cells are activated in the periphery and attracted by the particle to go to the lymph node. Dendritic cell attraction is a key aspect of vaccine work and cancer immunotherapy.

The system and method of the present invention employs hydrogel particle (NP or NT) enabled delivery and display of immune (dendritic) cell chemoattractants and purified concentrated breast cancer extracellular vesicle (EVs) cargo to the sentinel lymph node (SLN).

As such, it is the intent of the present invention to: a) facilitate SLN immune cell population remodeling and recruitment, b) provide for the migration of dendritic cells into the SLN to process the concentrated packages of EV cargo displayed on the particles, and c) effect immune priming and differentiation of the SLN dendritic cells, which in turn recruit CD8+Tcells. Therefore, the present invention is envisioned to test the approach in a murine syngeneic breast cancer model and a humanized mouse transplanted with human breast cancer. The measures of success are exhibited as a stimulated systemic immune recognition of the breast cancer resulting in a reduction of distant metastasis number and size. The proposed technology addresses the following roadblocks that have limited the success of immunotherapy for breast cancer:
1) Failure of the immune system to recognize the breast cancer cells as during tumor initiation and progression.
2) Cellular heterogeneity and molecular heterogeneity of breast cancer cell surface antigens that are presented to immune cells.
3) Failure to induce therapeutic immune cell infiltration of the tumor.
4) For current dendritic cell vaccine trials, failure of dendritic cell homing to the lymph node.
5) Absence of a method to harvest EVs from human tumor tissue micro-environment despite the fact that EVs shed into human tumor interstitial space would be an ideal source of tumor derived antigen for immune cell priming.

### Hydrogel Particle Chemistry

The system and method of the present invention employs porous open meshwork hydrogel particles (NTs or NPs) that can be programmed to passively capture and release a gradient of native chemoattractants, and EVs, using the new principle of controlled affinity release. A series of novel small molecule affinity synthetic dye ligands have been identified which bind proteins or nucleic acid molecules with incredibly high affinity. The affinity ligands are covalently immobilized in the hydrogel particles. The chemistry of the affinity ligands can be tailored to achieve a specific off-rate or a population of different off-rates.

The present invention employs hydrogel particle technology to address focused mechanistic questions about early events taking place within the SLN that can either suppress, or activate, the host immune recognition of the breast cancer. The present invention uses two different types of established murine host tumor models: 1) The 4T1 (originally spontaneously arising) breast carcinoma transplanted into its syngeneic host, and, 2) patient derived human breast cancer transplanted in a humanized mouse. Both of these models have been previously used to study immunotherapy or immune responses. The immunotherapy goal of this project is to use multi-function hydrogel particle technology to prime or awaken systemic immune recognition of the metastatic lesions distant from the SLN. To achieve the present invention, it was explored, in detail, the interaction of the hydrogel particles and their cargo within the cellular architecture of the SLN. For the 4T1 model the responding SLN immune cells are murine. In contrast, for the human breast cancer model the responding T-cells and dendritic cells are human. Under Aim 1, the system tests the efficacy of the multi-function particle treatment to reduce metastatic burden in an immunocompetent 4T1 syngeneic mouse model. Under Aim 2, the system optimizes a novel method to extract and purify interstitial fluid EVs from core needle biopsy for individualized immune priming. Under Aim 3 the system fluorescently labels EVs deriving from fresh patient interstitial tissue and from the MDA240 human breast cancer cell line, and the system characterizes EV trafficking and interaction with dendritic cells, T cells, and macrophages in vivo in the SLN of a humanized mouse model.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 depicts Cytokine array analysis of recipient cells. U937 monocytes were treated with either particles (NT) alone, HUT102 supernatants (HTLV-1 infected: 5 day) alone, or particles with bound HUT102 EVs (5 day HUT102 sup was rotated with particles overnight at 4° C.). All treatments were performed in duplicate. Treated U937 cells were incubated for 5 days, followed by harvesting of the cell supernatant and analysis via cytokine array according to the manufacturer's instructions. Controls of DI water and untreated U937 supernatant (5 day) were run on cytokine arrays simultaneously. Cytokines that were significantly upregulated with treatment of particles +HUT102 EVs in comparison to HUT102 sup alone are shown boxed in red in the lower left panel.

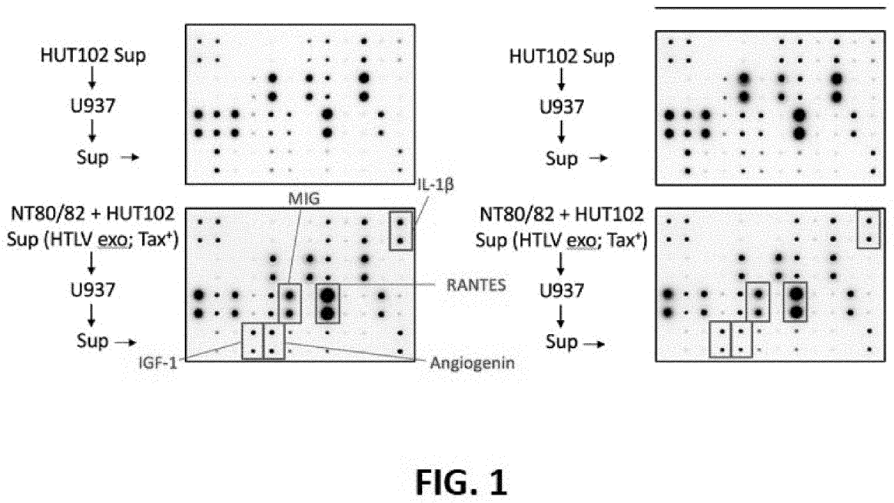

FIG. 2 exhibits Affinity particles (NT, NP, or hydrogel particles) which have captured intact cell derived EVs with high yield.

### DETAILED DESCRIPTION OF THE PREFERRED EMBODIMENT

The present specification discloses one or more embodiments that incorporate the features of the invention. The disclosed embodiment(s) merely exemplify the invention. The scope of the invention is not limited to the disclosed embodiment(s). The invention is defined by the claims appended hereto.

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment, Further, when a particular feature, structure, or characteristic is described in connection with an embodiment, it is submitted that it is within the knowledge of one skilled in the art to effect such feature, structure, or characteristic in connection with other embodiments whether or not explicitly described.

The system and method of the present invention validates, in vivo, a new hydrogel particle for the in vivo remodeling, and immune cell priming, of SLNs for research applications in cancer immunotherapy. The hydrogel particles employ a novel chemical affinity ligand to accomplish different synergistic functions within two different types of particles which home to the SLN. The first function is programmed release of chemoattractant or immune cell activating cytokines pre-loaded into the particles. The cytokines attract massive numbers of select immune cells, causing a radical remodeling of the sentinel lymph node (SLN) architecture and immune cell composition.

The second function is affinity capture and concentrated surface display of EVs pre-loaded onto the hydrogel particles. The hydrogel particle delivery and internalization of EV cargo causes significant activation of the host immune cell, in a manner distinctly different from the NP alone or the EVs alone. The particle technology are porous hydrogel particles constructed of cross-linked polymer network covalently coupled with affinity tuned dyes compounds, and surrounded by a cross-linked polymeric shell.

After loading with the cytokine or EV cargo, the particle technology is injected subcutaneously, or into the tumor parenchyma, upstream from the draining SLN. The particles are immediately carried by lymphatic drainage and home to the SLN where they induce a massive user pre-determined remodeling of the immune cell subpopulations within the SLN.

### Priming the Immune System to Recognize Breast Cancer in the SLN

An intact innate immunity is required for an anti-tumor immune defense. During neoplastic progression, cancer cell shed antigens, including vesicles from necrotic or apoptotic cancer cells, or whole cells, can enter the draining lymph and be carried to the SNL. The CD8 positive T cells infiltrating the tumor lesion are first programmed in the tumor draining SLN and then migrate to the local tumor stroma. The CD8 positive T cells, and other programmed immune cells that leave the sentinel node, migrate toward the gradient of shed EVs or tumor cells in the emerging breast tumor lesion. It has been hypothesized that emerging cancer evades detection by reprogramming the SLN, to initiate insidious cloaking of the emerging invasive breast cancer occurring upstream. Such tumor-derived immune suppression in breast cancer is evident in the reduced expansion of CD8+Tcells and other innate immune effector cells in the breast cancer SLN associated with invasion and aggressive behavior. The suppression of immune cell recruitment in the SLN by tumor cells is analogous to the suppression of the innate immune response to an infectious microorganism such as B. anthracis or toxoplasmosis. Toxoplasmosis organisms and anthrax spores suppress motility and connective tissue migration of recruited innate immune cells and dendritic cells during the incipient stages of infection. Virulence factors (i.e., ET and LT) from B. anthracis within the LNs retards the functions of immune cells such as dendritic cells (DCs), including their release of inflammatory CKs [36-39]. B. anthracis disrupts actin-based neutrophil migration in vivo, and toxoplasmosis blocks MMP activity preventing these cells from exerting their innate immune function [40]. By analogy, the system of the present invention proposes to reverse potential breast cancer SLN immune suppression using NP mediated delivery of chemoattractants and concentrated packages of EVs to prime, remodel, and pre-sensitize the SLN. Preliminary data supports this concept.

### Impact and Innovation

As shown in preliminary data, targeting to the SLN is highly specific and efficient and proceeds to attract a massive influx of selected immune cell populations.
The chemical principle of passive cargo delivery by affinity off-rate and the in vivo action is markedly different from previous particle technology LN drug delivery systems including liposomes, silicon or carbon particles, or antigen coated solid particles.
Controlled capture, display, and delivery of intact EVs to phagocytic cells, or to achieve direct in vivo T cell surface contact of the EV cargo has not been possible before.
The combination of local particle mediated chemoattraction of specific classes of immune cells with the delivery of EVs of known composition and size is a previously unexplored strategy for educating the tumor draining SLN.
This concept, and this technology, has broad flexibility for testing immunoprevention and immunotherapy strategies in preclinical models, and can provide insights for future clinical strategies.
The human breast cancer humanized mouse model will permit for the first time to study the human DCs and T cells recruited to the SLN following delivery of the chemoattractant and EV cargo.

### Hydrogel Affinity Particles (HPs NTs).

A series of novel small molecule affinity organic dye ligands have been identified that bind proteins, glycolipids, nucleic acids and therapeutic molecules with tunable affinity. The chemistries are immobilized on the hydrogel open mesh polymer. Cytokine nanoparticles (CKNP) passively (according to the off rate of the dye) release selected native chemoattractant proteins such as IL-8 and MIP-1α that call in specific subpopulations of immune cells, depending on the cytokine. The internal volume surface area of the particle is 1000 times greater than the surface area of a comparable solid particle, and the open particles freely exchange with the surrounding interstitial solute. The hydrogel particles are buoyant neutral density and are immediately carried to the lymph node where they can display or discharge their cargo in a passive means depending on the affinity of the capturing dye bait.

### Delivering Chemoattractants to the SLN for Immune Cell Priming

A variety of different classes of cytokines are well established to mediate chemoattractant or haptotactic recruitment of immune cells in the early stages of antigen challenge. It was hypothesized that the NP mediated delivery of a battery of chemoattractant cytokines to the SLN can override the suppression caused by factors elaborated from the primary tumor. Increasing the total number of SLN dendritic cells, macrophages, and granulocytes by cytokine priming can augment the readiness, and sensitivity, of the SLN to recognize the emerging tumor. Immune system recognition of the primary tumor is considered the primary factor determining the success of current clinical cancer immunotherapy trials using immune checkpoint inhibitors. Chemoattractant recruitment of dendritic cells may be an important attribute of the hydrogel particle technology. Roadblocks to immunization with antigen challenged dendritic cells has recently been reviewed. A major roadblock is failure of present strategies to induce the antigen pretreated dendritic cells to travel to the LN for antigen processing. The hydrogel particle technology overcomes this roadblock by recruiting activated dendritic cells to leave the skin and enter the SLN. The increase in dendritic cells alone may improve the immune recognition sensitivity for the tumor draining antigens into the SLN. As shown in the next section, it is proposed to first bring the dendritic cells and then the challenging tumor cell EV antigen to the SLN. This can synergize the potential benefit of the immunotherapy by massively enriching SLN immune cell recruitment, and then encouraging the recruited immune cells to engulf NPs/EV antigen.

### Delivering EVs to the SLN for Immune Antigen Training

The hydrogel particles offer a new opportunity to deliver and display pre-loaded EVs directly to the SLN. Compared to injection of native EVs into the subcutaneous space, NP transport of EVs, eliminates the many unknown variables of native EV penetration of extracellular matrix, lymphatic and vascular barriers, and uptake of EVs by lymphatic and vascular endothelium, on route to the SLN. Since the number of EV/EVs per hydrogel particle is known, and the NPs can be fluorescently labeled, the efficiency of SLN EV delivery can be quantified and locally correlated with the type of immune cells interacting with the NPs. Antigen coated on a particle is established to be much more efficient for dendritic cell activation, compared to the antigen alone. Delivering EVs via the hydrogel particles will achieve a higher local concentration of EVs in the SLN, thus isolating the research question to the role of the EV/EV in the SLN microenvironment. The type of EV secreted or discharged by cells is widely dependent on the cell state. Necrotic cells, apoptotic cells, and infected cells, for example, all elaborate different classes of EV composition and size. Challenging the SLN with different classes of EVs may have a profoundly different effect on the readiness of the SLN to recognize the tumor. It has been found that the EV size, and whether it is delivered by a hydrogel particle, can dramatically alter the mode of macrophage activation and cytokine production. Importantly, small sized EVs are relatively ignored by dendritic cells, unless the EVs are delivered by the hydrogel particle. Consequently, we will deliver to the SLN three different size classes of EVs spontaneously released murine 4T1 breast cancer or by human tumor cells, and then study the recruited classes of immune cells.

In support of this method, Cytokine particles induce massive remodeling of the SLN in 24 hours. SLN in vivo microenvironment chemoattractant remodeling. The cytokine particles (CKNP) are pre-loaded with native chemoattractant cytokines that attract a specific population of immune cells. The native cytokine retains full biologic activity and is slowly passively released from the open cage of the NP though the pores of the shell. The release rate is a function of the off-rate of the affinity dye, generating a controlled gradient of native chemoattractant molecules that attracts the responding immune cells to migrate toward the hydrogel particle. The loaded particles are injected sub-dermally or into the tumor where they immediately home to the regional draining SLN. The EV particles enter the SLN via the subcapsular sinus, are taken up by macrophages or dendritic cells and are transported to the cortex. During transit and arrest in the SLN, the CKNP release their chemoattractant cargo over 12 hours, causing the influx of immune cell subpopulations attracted by the cytokine. This strategy has been used via the present invention to successfully reverse the SLN innate immune suppression caused by infecting anthrax spores in mice. A single injection of the immune priming IL-8 and MIP-la particles dramatically and massively augmented recruitment and activation of immune cells to the SLN, compared to an absence of sustained recruitment using cytokines alone. The single injection prevented death in 70% of the mice cutaneously infected with B. anthracis compared to zero survival without the particle treatment. This documents the feasibility of the SLN priming/remodeling approach of the present invention.

### Lymph Node Remodeling

Fluorescently-labelled particle particles are quickly carried to the SLN when injected into the hind footpads of mice. The particles accumulate in the subcapsular and medullary regions of LNs, via lymphatic flow as well as phagocytic uptake by innate immune cells. Mice received injections of soluble, cytokines, blank, or preloaded (IL-8) particles into the hind footpads. At the specified times the number of cells in the SLN that stained positive for myeloperoxidase activity were counted from five randomly selected fields of view (0.002 mm2 each) at 100× magnification. Error bars correspond to 95% confidence intervals. * and # indicate p<0.05 between the corresponding counts with and without particles. Particle affinity release of cytokines induced elevated sustained neutrophil recruitment at 24 hours, not present using cytokine alone or blank particles. Although mice do not express CXCL8, they possess a receptor homologous to human CXCR2 that is able to mediate chemotaxis in response to human CXCL8.

CXCL8 causes high levels of immune recruitment in contrast to the endogenous mouse analogs MIP-2 and KC. Anticipating an increased combined effect of CKs belonging to different families, the hydrogel particles are loaded with the mouse CCL3 in addition to CXCL8. These chemokines were originally described as preferential chemoattractants and activators of mononuclear cells and eosinophils; and neutrophils. The cytokine loaded particles, following sub dermal foot pad injection were transported into the subcapsular sinus of the popliteal LN within 30 min. Beginning at 4 hours and continuing over 48 hours a massive 400 fold increase in recruited Ly-6G positive cells was noted causing swelling and enlargement of the node. Administration of cytokine loaded particles (CK-NP) result in the increased appearance and altered distribution of the neutrophil-specific antigen Ly-6G in the popliteal LNs of naïve and B. anthracis infected mice. Cytokine particle modification of the SLN is associated with pERK activation and migration of pERK activated dendritic cells toward the SLN Medullar region of popliteal LN in naïve mice or after injection of CKNPs for 28 h under 40× magnification. Immunohistochemical staining with pERK1/2− specific antibody. Overlapping patterns of immunohistochemical staining of LNs of mice injected into hind Footpads with NPs for 28 h. Primary antibodies against MHC II (C) and pERK1/2 (D). For each pair of images, consecutive slides of LN tissue were used and colorimetrically developed with diaminobenzidine (brown color, (C)) or Emerald Green (green color, (D)). Injection with CK-NPs activates tissue-resident Langerhans cells present at the site of injection and induces their migration to a deeper dermal location (and then to the SLN as dendritic cells). Administration of CK-NPs changes the distribution of MHC II pERK1/2+ cells at the site of injection (Footpads). pERK1/2+ cells in naïve and spore-challenged animals which did not receive CK-NPs were present as a distinct epidermal layer (left panels). Positively-staining cells in animals treated with CK-NPs were found migrated from their epidermal location to deeper dermal layers. Pre-treatment with CK-releasing particle (CK-NP) induces neutrophil and dendritic cell infiltration to reverse the bacterial-induced SLN chemotactic suppression, associated with increased survival of infected mice challenged in the FP with B. anthracis spores.

Hydrogel Particle Delivery of Cytokines Induces Immune Cell Infiltration in 4T1 Sentinel Lymph Node Micrometastasis.

Pilot experiments were conducted for which the timing and the lymph node examination were conducted in a manner analogous to the Anthrax experiment described above except that the mice were challenged in the foot pad with 4T1 cell line (10\hat{ }5). The team of investigators have long standing expertise of animal models of cancer metastasis. The 4T1 model system which is often used for experimental immunotherapy, generates a reliably high incidence of metastasis to the draining lymph node and distant organs. The feasibility studies indicate the following: a) 4T1 cell foot pad injection induces micrometastasis in the sentinel lymph node and lungs within 2 weeks in 4 out of 5 animals. b) pre-stimulation of the lymph node with cytokine delivery hydrogel particles induces a massive influx of granulocytes and dendritic cells within 48 hours in the BALB/c mice. c) induction of immune cell infiltrate to the location of 4T1 lymph node micrometastasis was noted and absent in the cytokine and particle alone control.

EV Affinity Particles Capture EVs and Display them to Monocytes. Purification of EVs Away from Other Particles Including Apoptotic Bodies.

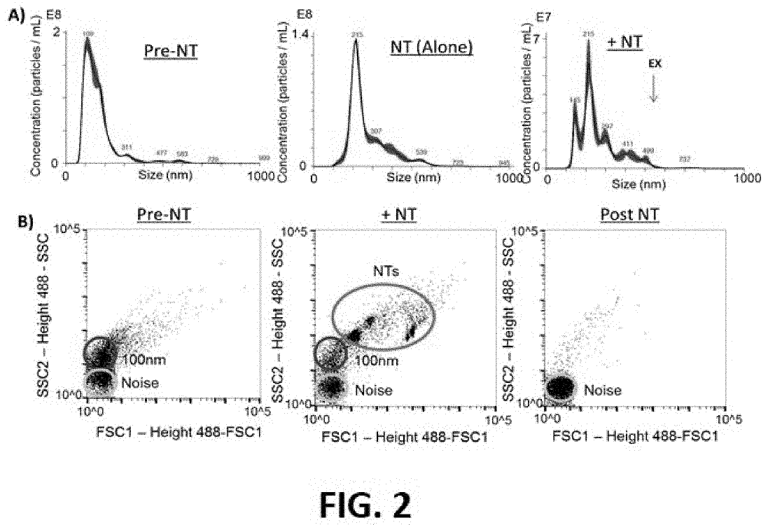

For cell culture supernatants, purification of EVs (<100 nm) begins with cell-free EV-containing fluids to which increasing centrifugal forces are applied [14, 60, 61]. The pellet is further purified over a gradient and characterized for EVs, potential contaminating virus or VLPs, or apoptotic bodies. It is routine to use several assays including EM, acetylcholinesterase (AChE) assay, qNano, and ExoELISA prior to functional analysis. EV affinity particles capture and retain intact EVs. After screening a large number of affinity dyes, a dye chemistry was identified that captured intact EV vesicles with high yield that led to complete depletion of EVs from cell supernatant of cultured cells. The captured EVs were intact as shown by laser capture of the Brownian motion of microvesicles and by nanoFACS. As seen in FIG. 2, there is a population of vesicles approximately 100 nm in size in tissue culture supernatants. After the addition of NP and EV cargo (NT) to the supernatant, these hydrogel particles could be visualized by a shift in FSC vs. SSC. After trapping by the NPs, the 100 nm vesicles (EVs) were removed from the tissue culture supernatant, leaving the post-particle sample absent of this population (FIG. 2, bottom right panel). As seen in FIG. 2 (top left panel), a population of vesicles has a mean size of 109 nm at a concentration of approximately 2×10⁹ vesicles/mL, hydrogel particles displayed an expected average size of 215 nm with a range in size from 200-500 nm (FIG. 2, top middle panel). When hydrogel particles were added to tissue culture supernatant, EVs were extracted from the media, as shown by an additional peak at −145 nm (FIG. 2, right panel) consistent with the AchE results that further supports that the vesicles isolated by these hydrogel particles were EVs. This supports capture via hydrogel particles as a method for EV isolation.

In short, FIG. 2 depicts Affinity particles which capture intact cell derived EVs with high yield. (A) Initial tissue culture supernatant from HTLV-1 infected cells prior to particle capture (Pre-NT) was analyzed for size and concentration by Nanosight. Pre-NT showed a population of 2×10\hat{ }8 109 nm vesicles along with 5×10\hat{ }8 311 nm, 477 nm and 583 nm vesicles. Hydrogel particles (NT Alone) showed a population of particles sized 215 nm-600 nm in size. Captured EVs were assessed after the particle capturing of initial tissue culture supernatant. Captured EVs (+NT) sample showed two major peaks representing both the NT and the EVs. (B) Initial tissue culture supernatants from HTLV-1 infected cells were analyzed by NanoFACS. Pre-NT shows a population of vesicles (circled in red) and a signal for noise, circled in yellow. In the middle panel (+NT), NT are circled in green and shown to be much larger than the 100 nm EV vesicles. The 100 nm vesicles, circled in red, are already showing a reduction in population after the addition of NTs. After capture of EVs, the left over supernatant (post-NT) shows that both the NTs and the EV vesicles population are now absent from the media.

Effect of NP/EVs Complex on Immune Cells

Next, it was tested whether hydrogel particles that bind to EVs could activate gene expression in the recipient cells. Either uninfected U937 EVs or infected HUT102 (HTLV infected cells) were treated with hydrogel particles and scored for cytokine gene expression using a filter array. The recipient cells (U937) were incubated for 5 days, sups were isolated and the cytokine array was run in duplicate. Results show that IGF-1, MIG, IL-lb, RANTES, and Angiogenin were all up regulated (2-7 fold) when EVs were complexed with hydrogel particles. This further indicates that many EVs (<100 nm) may not be recognized by the immune recipient cells (potentially as self), but when they are complexed with hydrogel particles, they are now being recognized (>100 nm) as non-self antigens which can activate the innate immune molecules leading to cell activation (i.e., M0 to M1 or M2 macrophages).

Therefore, per the previously disclosed tests, it is envisioned that the present invention can facilitate delivery of breast cancer EVs displayed on SLN homing particles along with particles releasing a dendritic cell chemoattractant will simultaneously recruit dendritic cells and specifically sensitize the immune recognition of the breast cancer. This induces systemic immune rejection of breast cancer colonies at a distance from the SLN, resulting in suppression or abolishment of micro and macrometastasis.

Additionally, it is envisioned that, per the system and method of the present invention, EVs can be harvested, and fully molecularly characterized, from the interstitial extracellular space of 36 freshly procured samples of individual patient human breast cancer tissue. Interstitial EVs, when delivered to the SLN of a humanized mouse model, in combination the particle technology described above, will recruit human CD8 positive T cells and human dendritic cells to the SLN. The proteome, exosome marker profile (CD63 and PD-L1), or size distribution, of the breast cancer interstitial exosomes from each patient will be distinct and this can be correlated with the propensity to induce human dendritic cell recruitment in humanized mice.

As such, it is an intention and ultimate result of the present invention to achieve multi-function particle (cytokine releasing and EV displaying) treatment to reduce metastatic burden in an immunocompetent 4T1 syngeneic mouse model (AIM 1). Further, it is the intent of the present invention to achieve optimization of a method for extracting and purifying EV from cultured cells and from fresh breast cancer interstitium (AIM 2). Finally, it is additional an intention of the present invention to provide for SLN remodeling by human breast cancer EVs in a humanized mouse model (AIM 3). It should be understood that the process and system of the present invention is envisioned to be applied to other diseases and cancers, and is not therefore limited to metastatic cancers.

Research Strategies

### Chemoattractant Particle (CKNP)/EV Display Particle (EVNP) Immune Cell Priming and SLN Remodeling

It was hypothesized that NP chemoattractant cytokines delivered to the lymph node in concert with particles that display breast cancer EVs will awaken the immune system in the SLN of a syngeneic animal model to recognize breast cancer cells transplanted upstream of the draining SLN. As such, it was expected that immune recruitment in the SLN to reduce the incidence of lymph node metastasis and reduce the systemic dissemination of the tumor. Under Aim 1 the present invention seeks to inject chemoattractant-releasing particles, and particles loaded with purified breast cancer EVs, into the footpad of mice, with, and without, subsequent (48 hours) challenging the footpad of syngeneic mice with 4T1 breast cancer cells. At 30 days all mice are euthanized following dissection of the lymph node and complete necropsy. The number and size of lymph node and major organ metastasis was then counted. The expectation is that priming the immune cell population of the lymph node combination immunotherapy will reduce the size and number of metastasis, will be associated with immune cell infiltration of the metastasis and will reduce the total body burden of the disease, compared to the following controls: 1) hydrogel particles alone, 2) cytokines alone, 3) saline alone, 4) EVs alone. As described below, the immune cell sub population composition of the lymph node and the immune cells infiltrating the metastasis will be evaluated by immunohistochemistry and flow cytometry, using methods previously established by the investigators' team.

Under Aim 2, the system of the present invention harvests and characterizes the EVs shed into the interstitium of freshly procured human breast cancer tissue using an optimized one step centrifugation/filtration method that does not rupture the tumor cells. For n=12 breast cancer samples, the full proteome of the interstitial EVs of three different types is characterized: 100K, 10K and 2K.

Under Aim 3, hydrogel particles are loaded with human breast cancer interstitial EVs and combine them with hydrogel particles preloaded with chemoattractant cytokines and study in vivo for modulation of SLN immune cell recruitment in the humanized mouse model. The Kashanchi lab has extensive experience with humanized mouse models including Rag/KO, NSG, and recently NOG animals. NOD/Shi-SCID/IL-2Rγc null (NOG) is an ideal in vivo mouse model to study ATL. The humanized breast cancer model is known to induce T cell recruitment and an antibody response. It is important to note that the immune cells infiltrate into the murine SLN following human breast cancer cell challenge into the foot pad of the humanized mouse will recruit human dendritic cells, macrophages and T cells. Since markers for human immune cell subtypes are well established, the histopathologic and flow analysis of the SLN accomplished via the present invention permits a novel investigation of particle interactions with human immune cells in a mouse model. The following methods re to be used to accomplish Aims 1-3:

### Choice of the Animal Models

Under Aims 1 and 3, the present invention employs a hydrogel particle approach to the 4T1 mouse model, and the human breast cancer in a humanized murine model.

The 4T1 mammary cancer model is well characterized, and has been used for preclinical testing of immunotherapy strategies. It has a predictable high rate of spread to local SLNs, followed by widespread organ metastasis, causing murine host lethality within three months. The time course of metastasis and progression is similar whether the 4T1 cells are transplanted in the mammary fat pad or into the foot pad of the BALB/c host. The present invention has established the cell number to implant in the foot pad to achieve reliable popliteal SLN micrometastasis within two weeks, with simultaneous pulmonary micrometastasis. 4T1 carcinoma cells can be clearly distinguished for enumeration of metastasis and infiltrating immune cells by IHC and flow cytometry. The 4T1 model offers the reproducibility and clinical relevance required to study strategies for stimulating the immune recognition of a spontaneous carcinoma in a syngeneic immune competent host. Finally, the present invention has established all the methods for studying the SLN immune response in the 4T1 model.

### Humanized Mouse Model

The human breast cancer humanized mouse model is a completely different type of model that can be transformative for breast cancer immunotherapy research. In the past few years, the SCID mutation that had been utilized in other models was crossed with the non-obese diabetic (NOD) mouse model and IL-2Rγc mouse resulting in an animal (NOG-SCID) that demonstrated a marked increase in tolerance and minimal GVH due to loss of virtually every innate immune cell. These animals could accept the xenotransplantation of blood cells forming fetal liver, bone, thymus, and lymphoid cells. Humanized mice are valuable small animal models are a valid model to study efficacy and mechanisms of cancer immunotherapy, but have not been applied previously to study breast cancer immunotherapy and have not been used previously to evaluate and optimize strategies for SLN immune priming by human immune cells. The relevancy of the NOG-SCID animal model to human cancer is two-fold: a) the tumor cells and the EVs are human and b) the immune cells that recognize the breast cancer EV immunization antigens are human immune cells derived from the mouse human stem cell reconstituted marrow. This offers a promising model for cancer immunotherapy. Nevertheless, the immunohistopathology characterization of the SLN draining node of the human breast cancer progression in the humanized model has not been previously published. Therefore, studies in the human breast cancer model will yield information about the feasibility of the new class of immunotherapy, and will also create a valuable model for testing future human breast cancer immunotherapy strategies.

Aim1. EV Isolation from 4T1 Cell Cultures and EV Characterization

It has been previously observed that EV of different sizes can be purified, characterized and captured by selected dyes covalently linked to the hydrogel particles. EV delivery by hydrogel particles to cultured monocytes stimulates cytokine production associated with macrophage activation and demonstrates that hydrogel particle delivery of EVs is highly potent for this effect compared to EVs alone or hydrogel particles alone. This is the rationale for the hypothesis that delivery of EVs to the sentinel lymph node following cytokine induction of immune cell influx educates the immune system to recognize the tumor associated antigens displayed by the EVs. EVs of different size classes are to be purified from cultured murine 4T1 cell line cells according to the methods shown in the preliminary data. Cell supernatant is preferably centrifuged at low speed to pellet cells and cell debris. The samples are then filtered to remove non-exosomal bodies, and ExoMAX reagent is added at a ratio of 1:1. Incubation occurs overnight at 4° C., which is followed by a low speed spin for 30 minutes to pellet the EVs. EVs are then further purified using OptiPrep™ gradient and hydrogel particles to capture and concentrate the EVs away from iodixanol. The EV preparation is visualized by TEM.

### Aim 1. Characterization of Particle Display of Cell Culture Generated EVs

The system and method of the present invention has been able to purify the EVs, however in this subaim, multiple particles are of focus, including vesicles less than 100 nm (classical EVs), more than 100 nm but less than 220 nm (EVs that contain pathogen associated proteins and non-coding RNAs), and larger than 220 nm which include ectosomes. It is important to note that under the conditions, EVs can be separated away from virus, VLP, and other extracellular vesicles such as exosome-like vesicles, ectosomes/microvesicles, and apoptotic bodies. For measuring EVs and ectosomes, several assays are routinely used, including EM, acetylcholinesterase (AChE) assay, qNano, and ExoELISA. The ExoELISA™ kit from System Biosciences binds exosome particles to a microtiter plate and a specific marker antibody (i.e. CD63). It has been consistently observed that CD63 is upregulated in EVs which helps to purify with either anti-CD63 beads or better recovery with hydrogel particles. Furthermore, a ZetaView™ is obtained to measure number, size, and membrane potential of vesicles, which utilizes hydrogel particle Tracking Analysis (NTA) based on Brownian motion as well as zeta potential analysis for the characterization of EVs. For the proposed experiments, particle capture and yield is quantified using NanFACs and FACS (anti-CD63).

### Aim 1. Particle Chemistry.

The process of the present invention synthesizes hydrogel neutral density open meshwork particles using poly(N-isopropylacrylamide) (pNIPAm) chemistry with N, N′-methylenebisacrylamide as a cross linker co polymerized with allylamine or acrylic acid (AAc) for incorporation of affinity ligands screened from a proprietary library of modified dyes coupounds. The hydrogel particles are characterized by their light scattering properties as described previously. The average particle diameter in PBS at 25° C. is in the range of 500 nm with an SD of 3 to 17 nm. The polydispersity index is 0.2-0.4 indicating a very low level of aggregation. The pore size is adjusted by crosslink frequency to allow diffusion of cytokines in the range of less than 25 kDa to freely exit the NP, or to select EV vesicle size exposure. Chemical dye derivatives (purity >95%) are coupled through the amino groups of the allylamine NP core. The chemical structure of the dyes contains multiple aromatic, condensed, and heterocyclic rings as well as 2 to 4 negatively charges sulfate groups per molecule. A library of 100 affinity dye chemistries has been created. These affinity ligands were found to bind molecules with a very high affinity (KD<10⁻¹³ M) and the affinity can be tuned downward by the side groups introduced into the molecule. Dye F3G-A, Reactive Blue, and modifications in the Trypan Blue molecule showed high affinity and low off rate for cytokines.

### Aim 1. In Vitro Characterization of Cytokine Release.

Cytokine uptake and affinity release are monitored in solution using ELISA and mass spectrometry. The loading efficiency for native cytokines at a concentration of 250 pg/mL ranges between 76% and greater than 95% depending on the cytokine and the affinity ligand. IL-8 (CXCL8) and MCP-1 (CCL2) release rate followed a typical mass action equilibrium binding and release kinetics release of >50% release in 10 h, with no albumin interference. CK-NP immune-stimulating potency is studied in vitro RAW 264.7 cells are exposed to MPs or controls which included serum-free DMEM/F12, LPS from E. coli. Blank particles and affinity dye in solution is used as controls. Supernatants are collected and analyzed using the Bio-Rad Bio-Plex Pro™ Mouse Cytokine 23-plex Assay for simultaneous determination of Eotaxin, G-CSF, GM-CSF, IFN-γ, IL-1α, IL-1β, IL-2, IL-3, IL-4, IL-5, IL-6, IL-9, IL-10, IL-12 p40, IL-12 p70, IL-13, IL-17A, KC, MCP-1, MIP-1α, MIP-1β, RANTES, and TNF-α. We have previously shown that we can load and slowly release in vivo MIP-1α and IL-8. Under this Aim we will extend the repertoire of cytokines and extend our analysis of immune cell subtypes in the SLN model. CCL3 (MIP-1α). CCL18 (MIP-4; PARC) CXCL8 (IL-8; KC; Gro-α) CXCL9 (MIG) and study the SLN recruitment in vivo of PMN leukocytes, T Cells Naïve, CD4+, CD8+, NK cells monocytes and dendritic cells.

### Aim 1. Animal Study Groups

The method of the present invention explores the timing and persistence of the SLN remodeling induced by cytokine releasing hydrogel particles in combination with purified 4T1 breast cancer EVs alone, or displayed by NPs, into the mouse mammary fat pad. The concentration gradient of the cytokine releasing particles will release greater than 1.0 ng of the chemoattractant per 4 hours over a 24 or a 48 hour period. Cytokine releasing particles will be injected in the foot pad at time zero. EV displaying particles will be injected at 24 hours. 1×10⁴ tumor cells will be injected at 48 hours. Mice will be sacrificed at 30 days after tumor challenge to study the lymph node micrometastasis and tumor progression. For this animal model the time of establishment of the disease is well known. Advanced disease is expected in all the untreated animals by 30 days after challenge. For the characterization subset we will harvest the SLN to evaluate the effect of the treatment on the immune cell composition, compared to controls. Experimental groups: A) Unloaded NPs; B), C), D) EV of different sizes 100K, 10K and 2K, respectively, E), F), G) particles loaded with 100K, 10K and 2K EV, respectively, H), saline control: N=96 total mice (8 groups with 12 mice per group). The best performing class of EVs (either 2K, 10K or 100K) will be studied with a time course experiment in presence and absence of particles: N=108 mice (3 groups with 12 mice per group, 3 time points). All mouse experiments are performed under an existing approved animal protocol at our AAALAC accredited university facility. The outcome is a direct test of the hypothesis that NP SLN priming in a syngeneic immune competent model will induce the number of tumor infiltrating immune cells and reduce the progression of the metastatic cascade and an assessment of the persistence of the immune SLN remodeling and the recruitment cell type.

Aim 2: Optimization of a Method for Extracting and Purifying EV from Cultured Cells and from Fresh Breast Cancer Interstitium

Exosomes and other EVs shed from the cell surface by breast cancer cells in vivo, within the tumor microenvironment, are immediately swept into the interstitial space and then carried by lymphatic draining to the SLN. In the past EVs have been harvested and characterized from cell culture supernatant or blood samples, not tumor interstitium. A fast and reliable method to extract EVs from fresh tumor tissue is urgently needed. We propose to optimize our novel method to extract interstitial fluid EVs based on tissue compression by slow speed centrifugation. Prior to application to human tissue samples, we will optimize the interstitial EV extraction conditions using the 4T1 cell line injected into a syngeneic mouse model (N=24 mice deriving from animal experiments in Aim 1). The method will yield rapid separation of EVs from the interstitial tissue without causing cell damage. We will optimize the method in terms of centrifugation speed, extraction buffer and filter molecular weight cut off. The parameters will be number of EVs and absence of apoptotic bodies indicating cell death. In order to achieve this goal, we will use tissue biopsies that produce EVs and suspend in 500 μl of PBS (without CA++/Mg++) and allow to sit at 4° C. for 1 hour. We will then take the mixture and pass it through a 0.22 μm filter using low speed centrifugation. This will allow most of the EVs to pass through the tissue and be collected at the bottom chamber (away from the tissue). As control, we will also use a 0.45 μm and 0.8 μm filter to look for larger (potentially apoptotic) or ectosomes which may contain DNA and histones (H2A, H2B, H3, and H4). The larger vesicles are not part of the exosome/ESCRT pathway and represent a very different set of proteins and nucleic acids. Therefore, focus is required to take the 0.22 μm filtered material and centrifuge at 2K, 10K, and 100K to isolate three distinct EVs. The 2K prep most likely will contain proteins that originated from autophagosome (secretory autophagy); 10K will contain a mixture of autophagy and exosome proteins originating from the endosome; and the 100K prep will contain pure EVs that contain tetraspanins (i.e. CD63, CD9, CD81) along with Alix and TSG101 proteins. We will define the proteomics content of the EVs as described below. We expect that we might obtain (depending on the size of the biopsies) at least 10¹⁰-10¹¹ EVs using this method which would be sufficient for proteomics characterization and animal studies of Aim 3. We will compare interstitium EVs with the EV derived from 4T1 cell lines and we will test the hypothesis that the in vivo EVs are different than in vitro. Interstitial EVs will represent heterogeneous cellular microenvironment which is a desirable characteristic for an effective immunogenic preparation. Future confirmation assays will look more in depth into the mechanism of signal transduction including RTKs at membrane, kinases that regulate NF-κB, gene expression of cytokines, and related read-outs of the recipient cells including cell cycle growth, apoptosis, and mitochondrial membrane alterations. Our future experiments will include defining the contents of the EVs including RNA-seq and metabolomics and functional assays in vitro (i.e. scratch test) and in vivo (wound healing test) in animals.

Aim 2. Mass Spectrometry (MS) Analysis of Interstitial Tissue EVs

EV suspensions in PBS will be reduced using 1% Rapigest SF surfactant (Waters) in 50 mM ammonium bicarbonate and 5% TCEP, alkylated using 50 mM iodoacetamide in 0 mM ammonium bicarbonate, and trypsin digested overnight. Digestion will be halted by adding trifluoroacetic acid to a final concentration of 0.1%. LC-MS/MS experiments will be performed on an Orbitrap Fusion (ThermoFisher Scientific, Waltham, Mass., USA) equipped with a nanospray EASY-nLC 1200 HPLC system (Thermo Fisher Scientific, Waltham, Mass., USA). Peptides will be separated using a reversed-phase PepMap RSLC 75 μm i.d.×15 cm long with 2 μm, C18 resin LC column (ThermoFisher Scientific, Waltham, Mass., USA). The Orbitrap Fusion will be operated in a data-dependent mode in which one full MS scan (60,000 resolving power) from 300 Da to 1500 Da using quadrupole isolation, will be followed by MS/MS scans in which the most abundant molecular ions will be dynamically selected by Top Speed, and fragmented by collision-induced dissociation (CID) using a normalized collision energy of 35%. “Peptide Monoisotopic Precursor Selection” and “Dynamic Exclusion” (8 sec duration), will be enabled, as will be the charge state dependency so that only peptide precursors with charge states from +2 to +4 will be selected and fragmented by CID. Peptide identification will be performed in Proteome Discover v 2.1 (Thermo Fisher) using trypsin constraints. Tandem mass spectra will be searched against the human UNIPROT database.

Aim 3: SLN Remodeling by Human Breast Cancer EVs in a Humanized Mouse Model

Activated DCs are required for priming naïve T cells either through cell-cell contact or by secreting their own EVs (DC EVs) that mature T cells, recruit inflammatory cells to the site of DC maturation (i.e., lymph nodes) for inflammatory cell recruitment. Therefore, in this aim, it is asked how EVs/hydrogel particles can potentially alter DC maturation and ultimately control T-cell maturation. It is hypothesized that EVs/hydrogel particles will efficiently mature DCs for either a physical contact and/or secret their own EVs that control recruitment of inflammatory cells. The ultimate goal is to observe whether hydrogel particle associated EVs (making the complexes larger than normal EVs; much like bacteria) can mature DCs much more efficiently. Also, when EVs are produced by DCs, it is expected that they can mediate the indirect activation of CD4⁺ T cells by presenting functional peptide-WIC complexes through a trans-dissemination mechanism. Under this Aim, we will load hydrogel particles with human breast cancer interstitial EVs of Aim 2 and combine them with NPs preloaded with chemoattractant cytokines (FIG. 3) and study in vivo for modulation of SLN immune cell recruitment in the humanized mouse model; we will use tumor tissue homogenates [76] from the same patient as a control. EVs will be fluorescently labelled to monitor in vivo trafficking. In order to fluorescently label the EVs, we will use the method described in FIG. 2. Briefly, EV suspension obtained as described in Aim 2 will be mixed with a 200 μM solution of the protein binding fluorescent dye 5-(and-6)-Carboxyfluorescein Diacetate Succinimidyl Ester in PBS. The dye binding reaction will be allowed to proceed for 2 hours at room temperature. EV preparation will be purified by size exclusion chromatography. 48 NOG-SCID humanized mice will be used for this aim. EV/NP+ cytokine NP (24 mice) and tumor tissue homogenates (24 mice) will be administered via injection in the foot pad as described in Aim 1. We will harvest the draining lymph node to evaluate the effect of the treatment on the immune cell composition, compared to controls, via fluorescent microscopy and immunohistochemistry.

### E.10.1 Human Tumor Tissue Procurement and Characterization.

Under Aim 3 we will procure fresh human breast cancer tissue from ongoing breast cancer surgery prior to therapy. The breast cancer tissue will be leftover not required for diagnosis. Multiple tissue replicates will be collected from each patient. We expect the tumor grades and histologies of the breast cancers donated to be a representative sampling of the human breast cancers seen at any major community hospital, including a high proportion of minority and underserved communities served by the Sentara Hospital system. Such diversity is an important part of our study. For this project our Aim 3 goal is the following
A. Characterize the size distribution and the full proteome, and the exosome marker profile of human breast cancer tissue interstitial exosomes (FIG. 4) within patients (five replicates) and between patients (n=36). This includes the PD-L1 and CD63 marker distribution by nanoFlow.
B. For each patient for the three size classes of EVs evaluate the SLN remodeling in the Humanized mouse model.
C. Correlate the histopathology, PD-L1 scoring, immune cell composition, ER, PR, HER2, and Ki67 score of the patient's primary tumor donated specimens with the outcome within the SLN. Test the hypothesis that specific characteristics of the patient tumor interstitial exosomes, and SLN remodeling response, correlates with the patient histopathologic scores.

### E.11 Statistical Methods:

The variables follow distributions from nominal to interval such as number of immune cells recruited, number of metastases or survival time. This will require multivariable statistical models. In addition to exploring bivariate associations between lymph molecular content and outcome variables using appropriate statistical tests (t-test, Spearman's rho correlation, Fisher's Exact), multi-variable models will be constructed using logistic regression or a mixed model where appropriate. A mixed model will be used where some predictor variables are repeated measures at different times or with cases having an unequal number of evaluations. All analysis will assume a 2-tailed alpha=0.05, based on a power calculation of n=12.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

We claim:

1. A method of harvesting extracellular vesicles for delivery to, and remodeling of, an immune system comprising:
subcutaneously injecting hydrogel particles with cytokine to remodel the lymph node;
delivering EVs via the hydrogel particles to achieve a higher local concentration of EVs in the SLN;
the EV particles entering the SLN via the subcapsular sinus;
macrophages/dendritic cells take up the EV particles and transport them to the cortex;
CKNP releasing chemoattractant cargo causing an influx of immune cell subpopulations attracted by the cytokine; and
reversing the SLN innate immune suppression.
